(19) United States
(12) Patent Application Publication
(10) Pub. No.: US 2021/0402019 A1
(43) Pub. Date: Dec. 30, 2021

(54) UVC LIGHTING SYSTEM

(71) Applicant: Gregg L. Rosenbaum, Wellington, FL (US)

(72) Inventor: Gregg L. Rosenbaum, Wellington, FL (US)

(21) Appl. No.: 16/915,460

(22) Filed: Jun. 29, 2020

Related U.S. Application Data
(60) Provisional application No. 63/043,664, filed on Jun. 24, 2020.

(51) Int. Cl. A61L 2/10; A61L 2/26

(52) U.S. Cl. CPC A61L 2/10; A61L 2202/11; A61L 2/26

(57) ABSTRACT

Methods and apparatus are disclosed for emitting UVC light at a wavelength that is safe for human exposure, and effective to kill pathogens. The apparatus includes a UVC lighting system that is configured to emit the UVC light.

### CROSS-REFERENCE TO RELATED APPLICATIONS

This claims priority to U.S. Patent Application Ser. No. 63/043,664 filed Jun. 24, 2020, the disclosure of which is hereby incorporated by reference as if set forth in its entirety herein.

### BACKGROUND

Viruses can be highly contagious, and can pose numerous health risks, up to and including death. For instance, COVID-19 has had a dramatic effect not only on peoples' lives individually, but on the global economy as a whole. For instance, current estimates are that approximately nine million people have contracted COVID-19 to date across the globe, and approximately five hundred thousand people have died. Many countries, including the United States, mandated the closure of all businesses not deemed to be essential. As a result, many small businesses were forced to close, in some cases permanently. According to sources, airline travel dropped 96 percent due to COVID-19 as of April, 2020. Professional sports leagues have postponed ongoing seasons, and delayed the opening of seasons. Music and other concerts have been postponed or cancelled. Office buildings have been evacuated.

As localities push to normalize the economy, health officials continue to grapple with best practices to minimize transmission of the virus in areas of public gathering. To date, no vaccine exists to counteract COVID-19, no cures exist for those seriously ill with the virus, and social distancing remains the most recommended course of action to reduce the likelihood of contracting the airborne virus. However, many businesses are unable to return to normal operation under social distancing guidelines. Moreover, while social distancing can reduce the odds of contracting an airborne illness, it does not prevent contraction of the airborne illness. Further, social distancing does not prevent people from contracting the virus by coming into physical contact with a contaminated surface, and subsequently ingesting the virus through the mouth, nose, or eyes.

While COVID-19 has been the topic of the headlines in recent days, it does not escape notice that tens of thousands of people die from influenza each year. For instance, eighty thousand people died from influenza in 2018, according to the Centers for Disease Control and Prevention. Therefore, even if medical science were to eliminate COVID-19 as an immediate threat, other viruses will remain potent and lethal. Further, other pathogens, some known and others unknown, will remain a threat.

Therefore, what is urgently needed is a method and apparatus for eliminating contagions, including COVID-19 and other contagions including bacteria, viruses, and the like.

### SUMMARY

In one example, a UVC lighting system can include an optical cable including an optically transmissive side emitting core that is elongate along a central axis, and an outer sheath that surrounds the core. The system can further include a UVC light source configured to emit UVC light at a wavelength that is in a range from 208 nm to 254 nm, wherein the UVC light source is coupled to the optical cable such that at least a portion of the UVC light emitted from the UVC light source is directed into the optical cable. The core is configured to receive the emitted UVC light from the UVC light source, and emits the light through the outer sheath in a direction angularly offset with respect to the central axis.

### BRIEF DESCRIPTION OF THE DRAWINGS

The foregoing summary, as well as the following detailed description of illustrative embodiments of the intervertebral implant of the present application, will be better understood when read in conjunction with the appended drawings. For the purposes of examples of the present disclosure, there is shown in the drawings illustrative embodiments. It should be understood, however, that the application is not limited to the precise arrangements and instrumentalities shown. In the drawings:

FIG. 1A is a schematic view of a UVC lighting system constructed in accordance with one example, including a UVC light source and a UVC emitter configured as a side emitting optical cable;

FIG. 1B is a sectional perspective view of a portion of the UV-C optical cable illustrated in FIG. 1A;

FIG. 1C is a sectional perspective view of the portion of the UV-C optical cable illustrated in FIG. 1A in another example;

FIG. 2 is a perspective view of a reel of optical cable illustrated in FIG. 1A;

FIG. 3A is a perspective view of UVC lighting system of FIG. 1A, including a support structure that supports the optical cable in one example;

FIG. 3B is an exploded perspective view of a portion of the support structure and a portion of the optical cable of FIG. 1A;

FIG. 3C is a sectional elevation view of the support structure illustrated in FIG. 3A.

### DETAILED DESCRIPTION

The present disclosure recognizes the ultraviolet (UV) light can be effective to eradicate pathogens including bacteria such MRSA and other bacteria, and other pathogens that are exposed to the light, such as viruses including the influenza virus, the COVID-19 virus, and other viruses. However, many types of ultraviolet are unsafe to humans and other animals. UVC light, on the contrary, includes wavelengths in its spectrum that are safe for human and animal exposure, and also provide the benefit of eradicating pathogens that are exposed to the UVC light. For instance, UVC light having wavelengths within a wavelength range from approximately 208 nm (nanometers) to approximately to 254 nm are believed to be safe for human exposure. Further, UVC light in the wavelength range can effectively neutralize bacteria, viruses, and other pathogens that are exposed to the light.

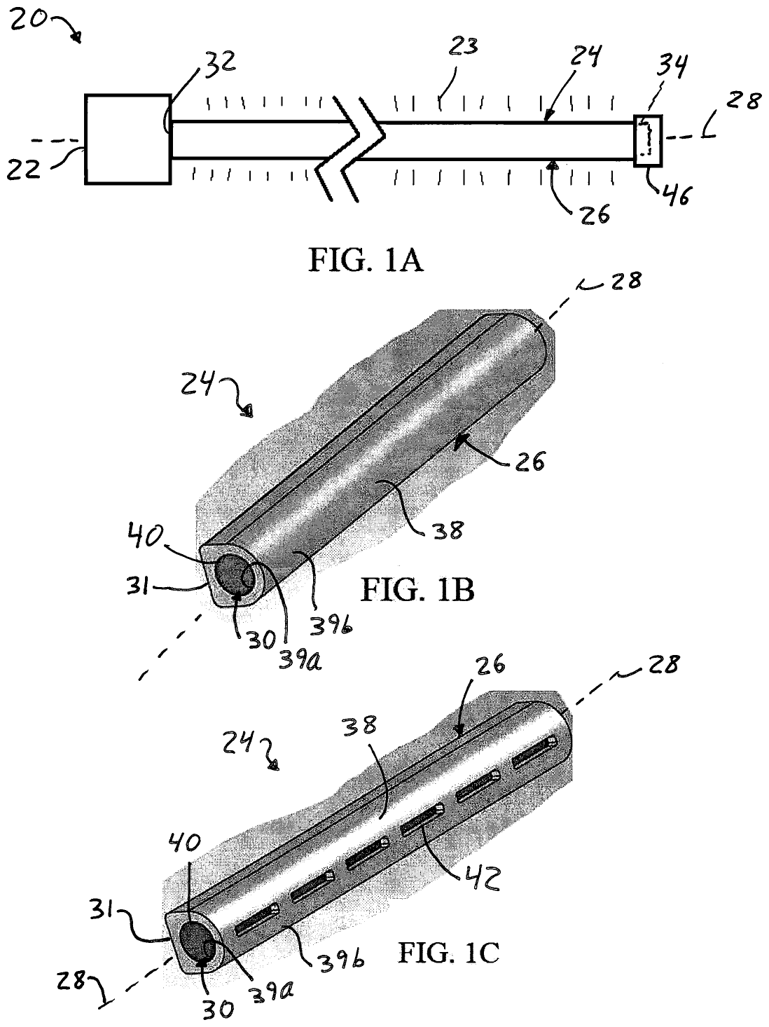

Referring now to FIGS. 1A-1B, a UVC lighting system 20 constructed in one example includes at least one UVC light source 22 and at least one UVC emitter 24 that is in optical communication with the at least one UVC light source 22. The UVC light source 22 can emit far UVC light 23 at a desired wavelength and wattage. The UVC emitter 24 can be configured as an optical cable 26 that can be mounted to any suitable structure or apparatus as desired. The UVC emitter 24 is configured to be coupled to the UVC light source 22, such that the UVC light source 22 directs UVC light into the emitter 24, which then emits the UVC light 23 received from the UVC light source 22.

The optical cable 26 can be configured as a side emitting optical cable 26. That is, the optical cable 26 can be elongate along a central axis 28, and the optical cable 26 can be configured to emit light along a direction angularly offset with respect to the central axis 28. For instance, the optical cable 26 can be configured to emit light along a direction substantially perpendicular to the central axis 28.

In one example, the optical cable 26 includes an optically transmissive core 30 that is elongate along the central axis 28. The optically transmissive core 30 is configured to receive UVC light emitted by the UVC light source 22 when the optical cable 26 is coupled to the UVC light source 22. For instance, the optical cable 26 defines a proximal end 32 and a distal end 34 opposite the proximal end 32 along the central axis 28 in a distal direction. The proximal end 32 can be coupled to the UVC light source 22 using any suitable connector as desired that is configured to direct light from the UVC light source 22 to the optical cable 26.

The optical cable 26 can include a substantially optically transparent core 30 that extends along the central axis 28, and an outer sheath 38 that surrounds the optically transparent core 30. In particular, the outer sheath 38 also extends along the central axis 28 and surrounds the optically transparent core 30 along a plane that is substantially perpendicular to the central axis 28. In particular, the outer sheath 38 has an inner sheath surface 39a that faces an outer side 37 of the core 30, and an outer sheath surface 39b opposite the inner sheath surface 39a. The terms “substantially,” “approximately,” “about,” and derivatives thereof as used herein with respect to a parameter (e.g., shape, size, angle, dimension, direction, and the like) includes the stated parameter and parameters within +/−10%, such as +/−5%, for instance +/1 2%, including +/−1% of the stated parameter.

In one example, the optical cable 26 can be flexible and thus configured to attach to any suitable structure in any suitable path as desired. The optical cable 26 can define an attachment surface 31 configured to be mounted to any suitable surface of the support structure. In one example, the surface 31 can be substantially planar both along a direction parallel to the central axis 28, and along a second direction perpendicular to the central axis 28. The surface 31 can be defined by the outer sheath 38, and in particular the outer sheath surface 39b. The remainder of the outer sheath surface 39b can extend along any suitable path as desired in a plane that is perpendicular to the central axis. For instance, the remainder of the outer sheath surface 39b can extend along a circular path, an oval-shaped path, or any polygonal path as desired. Alternatively, the surface 31 can be defined by a bracket that is attached to the optical cable 26. The surface 31 can be adhesively attached to the support structure, attached using hooks and loops, or attached using any suitable alternative mechanical fasteners as desired.

The optically transparent core 30 can include at least one side emitting fiber 40 that be any suitable flexible optically conductive material. In one example, the material of at least one optical fiber 40 can be a polymethylmethacrylate (PMMA) material, though it should be appreciated that the material of the at least one optical fiber can be any suitable alternative material as desired. The core 30 can include a single optical fiber 40 in some examples. In other examples, the core 30 can include a plurality of optical fibers 40. For instance, when the core 30 includes a plurality of optical fibers 40, the optical fibers 40 can be braided so as to define a braided structure. While PMMA allows the core 30 to be flexible in the manner described above, it is envisioned in other examples that the core 30 can be rigid in some applications. In this regard, the core 30 can be glass, or any suitable alternative rigid light propagating material as desired.

The sheath 38 can be any suitable polymeric material that is substantially optically transparent. For instance, the material of the outer sheath 38 can be a polyethylene, though it should be appreciated that the material can be any suitable material as desired. The polyethylene can be substantially optically transparent, such that light emitting out the side of the core 30 can pass through the material of the outer sheath 38 from the inner sheath surface 39a to the outer sheath surface 39b.

In some examples, referring to FIG. 1C, it is envisioned that one or more apertures 42 can be created in the sheath 38 that extend from the inner sheath surface 39a to the outer sheath surface 39b. The apertures 42 can be spaced from each other along a direction parallel to the central axis 28. Further, the apertures 42 can be elongate along the direction that is parallel to the central axis. The apertures 42 can further be aligned with each other along the direction parallel to the central axis 28. The apertures 42 can be arranged in one or more rows that are oriented along the direction parallel to the central axis 28. The apertures 42 can be arranged in a regular pattern a desired. For instance, the apertures 42 equidistantly spaced from each other. Alternatively, the apertures 42 can be irregularly arranged, and can thus be variably spaced from each other as desired. The apertures 42 can have a substantially identical size and shape, or can be differently sized and/or shaped as desired. Without being bound by theory, it is believed that light emitted out the side of the core 30 can travel unencumbered through the apertures 42.

Referring now to FIGS. 1A-1C, the core 30 and the outer sheath 38 can have any suitable dimensions as desired. For instance, the core 30 can have a diameter (or other cross-sectional dimension that extends through the central axis if the core does not have a circular cross-section) that is in a range from approximately ¼ inch to approximately ¾ inch, such as approximately ½ of an inch. It is recognized, of course, that the core 30 can define any suitable alternative cross-sectional dimension as desired. The outer sheath 38 can have a thickness from the inner sheath surface 39a to the outer sheath surface 39b that can be less than the cross-sectional dimension of the core 30. In one example, the thickness of the outer sheath 38 can be in a range from approximately 1/50 inch to approximately ¼ inch, including approximately ⅛ inch. It is recognized, of course, that the sheath 38 can define any suitable alternative thickness as desired. In one example, the sheath 38 can be co-extruded with the core 30. In other examples, the sheath 38 can be extruded, such that the inner sheath surface 39a defines an opening, and the core 30 is inserted into the opening.

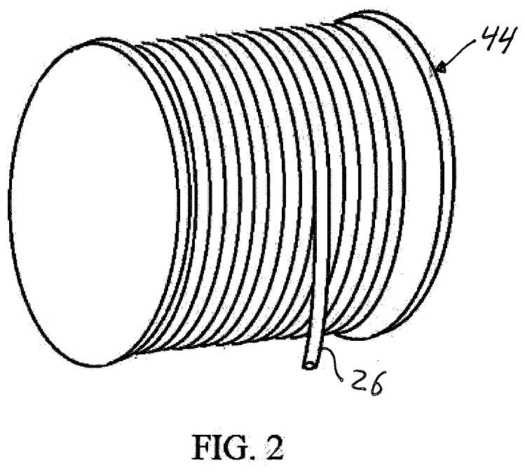

Referring now to FIGS. 1A-2, and as described above, a length of the optical cable 26 can be coupled to the UVC light source 22. In one example, the optical cable 26 can be rolled onto a reel 44, and subsequently fed to a desired length and cut at that length so as to define a length of cable 26 having the proximal end 32 and the distal end 34 as described above. The proximal end 32 can be coupled to the UVC light source 22 such that, when the UVC light source 22 is activated, UVC light directed from the light source 22 to the optical cable 26 travels along the core 30 from the proximal end of the core to the distal end of the core that is spaced from the proximal end of the core in the distal direction. As the UVC light propagates through the core 30 from the proximal end to the distal end, light is also emitted out the side 37 of the core 30. Light is thus emitted from the optical cable 26 into the ambient environment. The optical cable 26 can include one or more light shaping elements that are attached to the outer sheath 38, such that the UVC light travels through the outer sheath 38 and through the light shaping element as it is emitted from the optical cable 26. In one example, the at least one light shaping element can be configured as a diffuser, a lens, or any suitable alternative light shaping element as desired. Further, light shaping elements can be disposed at any suitable location as desired. For instance, the light shaping elements can cover the apertures 42 in one example.

The UVC lighting system 20 can further include a cap 46 that is configured to be attached to the optical cable 26 to cover the distal end 34. In particular, the cap 46 can be press fit or otherwise attached to the optical cable 26, and in particular to the outer sheath 38, such that the cap 46 covers the core 30 at the distal end 34. The cap 46 can be optically opaque so as to prevent UVC light from being emitted out the distal end 34 of the cable 26 along a direction substantially parallel to the central axis 28. Thus, the core 30 can be closed at the distal end 34 of the cable 26. It is appreciated that any suitable alternative method and apparatus can be used to close the core 30 at the distal end 34 as desired.

The light source 22 can emit the UVC light at any suitable wattage as desired, depending on the area of the UVC light coverage desired in which the UVC light will be effective to eliminate pathogens. In general, higher wattage levels eliminate pathogens in larger coverage areas. In some examples, the wattage can range from as little as approximately 4 watts to approximately 10 watts, including from approximately 6 watts to approximately 8 watts. In other examples, the wattage can be greater than 10 watts, and can range up to approximately 100 watts or more, including hundreds of watts. The light source 22 can further emit the UVC light is emitted at a wavelength that is safe for human exposure. Thus the wavelength can be in a range from 208 nm to 254 nm. In one example, the wavelength is approximately 222 nm, though it is recognized that the wavelength can be any suitable wavelength that is effective at killing pathogens exposed to the UVC light and safe for human exposure. The light emitter 24 can be positioned at virtually any location where it is desired to eliminate pathogens from the ambient environment to increase public safety for those disposed at the location, particularly at locations where social distancing is impractical. Examples of suitable locations can include, without limitation, stadiums, office buildings, nursing homes, lavatories, hotels, schools, movie theaters, airplanes, airports, trains, residences, social gatherings, or any location populated by humans. Thus, the ambient environment can be indoors or outdoors. Advantageously, the UVC light 23 is safe for human exposure, and thus the UVC lighting system 20 can be activated in the presence of humans.

Further, light emitters 24 can be placed at different locations within an environment, such that some of the light emitters 24 can emit the UVC light to pathogens disposed in shadows created by other light emitters 24. When the light emitter 24 is configured as the optical cable 26, the optical cable is flexible such that the cable 26 can be directed along any suitable path as desired. For instance, the cable 26 can be bent at least up to 90 degrees at a bend location without preventing the UVC light from traveling through the core 30 past the bend location. The optical cables 26 can also define any suitable length as desired from the proximal end to the distal end, including several inches less than a foot, up to several feet, over ten feet, tens of feet, hundreds of feet, or more, depending on the architecture of the UVC lighting system 20.

The light emitters 24 can each be coupled to their own dedicated UVC light source. In other examples, multiple light emitters 24 can be coupled to a single UVC light source. Further, it is envisioned in some examples, that the UVC lighting system can be portable and handheld to eliminate pathogens in the immediate vicinity of the user. For instance, the UVC lighting system can be a wearable technology, and supported by clothing such as shirts, pants, jackets, footwear, hats, belts, and the like. It is envisioned that the UVC system can be worn by military personnel to reduce or prevent the spread of pathogens for those who are in close quarters with each other. In this regard, the UVC light source 22 can receive electrical power from any suitable power source as desired. In one example, the UVC light source 22 can be battery powered in some examples. The battery can be rechargeable. For instance, the UVC light source 22 can be powered by a lithium ion battery, or any suitable alternative battery as desired. In other examples, the UVC light source 22 can be plugged into a conventional electrical receptacle, and receive electrical power from an electrical power grid.

At least a portion of the UVC light emitted from the UVC light source 22 is directed into the optical cables 26. For instance, if each optical cable 26 is coupled to its own dedicated light source 22, then a substantial entirely of the light produced by the UVC light source 22 is directed to the optical cable 26. If a plurality of optical cables 26 are coupled to a common UVC light source 22, then a portion of the UVC light from the UVC light source 22 is directed into each of the optical cables 26.

The optical emitter 24 can expose pathogens disposed in the ambient environment to the UVC light, such that pathogens sensitive to the UVC light can be eliminated. The pathogens can be airborne pathogens, or can be disposed on a surface. Thus, the optical emitter 24 can expose pathogens that are airborne in the ambient environment and that are disposed on a surface in the ambient environment to the UVC light. It is believed that the COVID-19 virus is sensitive to the UVC light, and thus can be eliminated upon exposure to the UVC light.

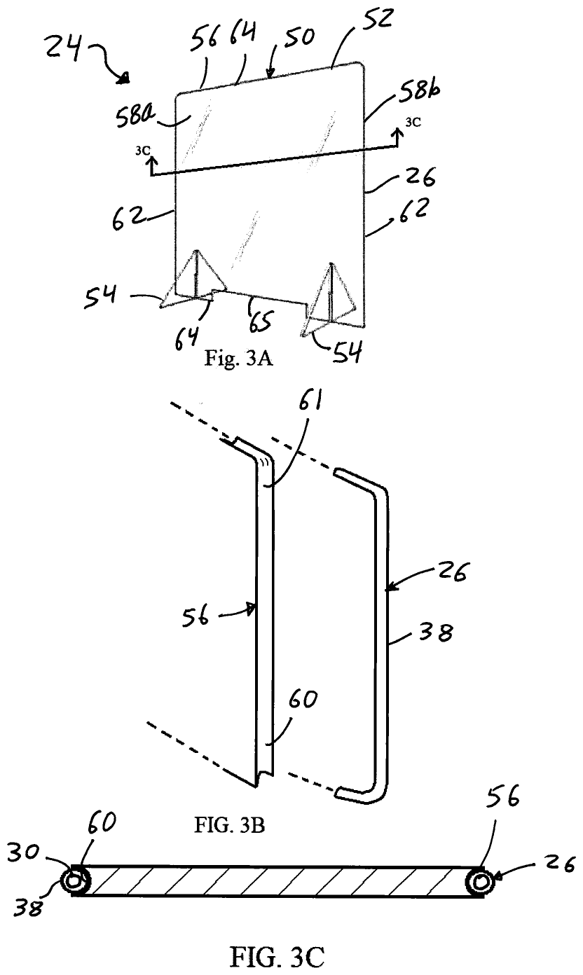

While the optical emitter 24 can be configured as a cable in the manner described above, it is recognized that the optical emitter can be configured as any suitable apparatus configured to receive UVC light from a UVC light source, and emit the UVC light in a desired direction. Referring now to FIGS. 3A-3C, in other examples, the optical emitter 24 can further include a support structure 50. The support structure 50 can be configured as a wall 52 that is configured to support the optical cable 26 to emit the UVC light in the manner described above. In one example, the wall 52 can be placed between people or groups of people, and thus can be referred to as a divider wall. The wall 52 can be made of glass, plexiglass, or any alternative suitable material. Thus, in some examples, the wall 52 can be optically transparent to allow line of sight through the divider wall. The wall 52 can be supported by any suitable stand 54 that can rest against a support surface, or can be otherwise supported as desired. The wall 52 can support the optical cable 26 in any suitable manner. In one example, the optical cable 26 can extend about at least a portion up to an entirety of an outer perimeter 56 of the wall 52. The outer perimeter 56 can extend between front and rear farces 58a and 58b of the wall 52. The front and rear faces 58a and 58b can be planar or alternatively shaped as desired. Further, the front and rear faces 58a and 58b can be defined by the same single pane (for instance of plexiglass), or can be defined by first and second panes, respectively. Thus, in other examples, the optical cable 26 can be disposed between the first and second panes in alternative examples.

The optical cable 26 can be mounted to the outer perimeter 56 in any suitable manner as desired. For instance, the outer perimeter 56 can define a concavity 60 in cross-section, and the optical cable 26 can nest or otherwise be at least partially disposed inside the concavity 60. The support structure 50 can include a pair of opposed sides 62 and opposed ends 64 that extend between the sides 62. The concavity 60 can extend along a portion up to an entirety of any one or more up to all of the opposed sides 62 and the opposed ends 64. The bottom end 64 can define a notch 65. The UVC light source can be disposed on the support surface and extend into the notch 65 as desired. Thus, one or more lengths of the optical cable 26 can extend along a portion up to an entirety of one or more up to all of the opposed sides 62 and the opposed ends 64. In this example, the outer sheath 38 of the optical cable 26 can be cylindrical, and thus can be constructed so as to not include the planar surface described above. In other examples, it is appreciated that the outer perimeter 56 can define a substantially planar surface, and thus the substantially planar surface of the optical cable 26 can be attached to the planar surface of the outer perimeter 56 in the manner described above. While the wall 52 can be configured as a divider wall in some examples, it is appreciated that it can be placed anywhere, and not necessarily between people or groups of people. For instance, the support structure 50 can be placed in a hallway, entryway, or common area to emit the UVC light therein. Alternatively, the wall 52 can be mounted onto a building wall, or can be suspended by the ceiling. In additional examples, one or both of the front and rear farces 58a and 58b can carry advertisement or other message-containing media, such as a menu, schedule, or the like.

Methods for eliminating pathogens using the UVC lighting system 20 are also contemplated. For instance, a method of eliminating pathogens from an ambient environment can include the step of coupling the proximal end 32 of at least one side-emitting flexible optical cable 26 to the UVC light source 22, and attaching a length of the cable 26 to a support surface. The support surface can be defined by the support structure 50 in the manner described above. The attaching step can include routing the optical cable along one or both of a linear path and a non-linear path. The method further includes the step of directing the UVC light 23 from the UVC light source 22 into the optical cable 26, such that the UVC light 23 travels along the core 30. The UVC light 23 has a wavelength that is in a range from 208 nm to 254 nm. The method can further include the step of emitting the directed UVC light 23 from the optical cable 22 to the ambient environment along a direction angularly offset with respect to the length, which can be measured along a direction that is substantially parallel to the central axis 28. It should be appreciated that the steps of coupling, attaching, and activating can be performed in any order.

The UVC light 23 is emitted from the optical cable 26 in sufficient amount to kill pathogens in the ambient environment that are exposed to the UVC light 23. The pathogens can include viruses such as COVID-19 viruses, influenza a, influenza b, and others, and bacteria such as MRSA and others. The coupling step can include coupling the proximal end 32 of the optical cable 26 to the light source 22. The method can include preventing the directed light from being emitted from the distal end 34 in a direction substantially parallel to the length, for instance by attaching the cap 46 to the distal end 34. The method can further include the step of cutting the length of optical cable 26 from the reel 44. The method can further include directing UVC light to a plurality of optical cables 26. The proximal ends 32 of the optical cables 26 can be coupled to respective different light sources 22, such that a substantial entirety of the light emitted by each of the light sources 22 is directed into the coupled optical cable 26. Alternatively, the proximal ends 32 of the optical cables 26 can be coupled to the same light source 22, such that each of the optical cables 26 receives a portion less than a substantial entirety of the UVC light that is emitted by the UVC light source 22.

It should be appreciated that the UVC lighting system 20 can be installed at any location where it is desired to eliminate pathogens. The location can be populated by humans, such that the UVC light 23 can eliminate pathogens in real time that are introduced into the air, for instance due to breathing, sneezing, and coughing. Further, the UVC light 23 can eliminate pathogens being left on surfaces. Thus, people can congregate in the presence of the UVC light 23 at the location while being subjected to reduced risk of catching the pathogens from other people at the location, even from other people in close proximity.

It should be understood that the foregoing description is only illustrative of the present invention. Various alternatives and modifications can be devised by those skilled in the art without departing from the present invention. Further, it should be appreciated that all features of all examples disclosed herein can be incorporated into all other examples unless otherwise indicated. Accordingly, the present invention is intended to embrace all such alternatives, modifications, and variances that fall within the scope of the appended claims.

What is claimed is:

1. A UVC lighting system comprising:
an optical cable including an optically transmissive side emitting core that is elongate along a central axis, and an outer sheath that surrounds the core; and
a UVC light source configured to emit UVC light at a wavelength that is in a range from 208 nm to 254 nm, wherein the UVC light source is coupled to the optical cable such that at least a portion of the UVC light emitted from the UVC light source is directed into the optical cable,
wherein the core of the optical cable receives the emitted UVC light and emits the light through the outer sheath in a direction angularly offset with respect to the central axis.

2. The UVC lighting system of claim 1, wherein the outer sheath is an optically transparent material.

3. The UVC lighting system of claim 2, wherein at least a portion of the emitted UVC light travels through the material of the outer sheath.

4. The UVC lighting system of claim 1, wherein the optical cable is flexible so as to bend at least up to 90 degrees at a bend location without preventing the UVC light from traveling past the bend location.

5. The UVC lighting system of claim 4, wherein the outer sheath defines an inner surface that faces the core, and an outer surface opposite the inner surface, wherein the outer sheath defines at least one aperture that extends from the inner surface to the outer surface, so that at least a portion of the emitted UVC light travels through the at least one aperture.

6. The UVC lighting system of claim 1, wherein the cable further comprises a substantially planar surface configured to mount to a support structure, such that light emitted from the cable is emitted in a direction away from the support structure.

7. The UVC lighting system of claim 6, further comprising the support structure.

8. The UVC lighting system of claim 7, wherein the support structure comprises front and rear farces and a perimeter that extends between the front and rear faces, and the optical cable is mounted to the perimeter.

9. The UVC lighting system of claim 8, wherein the perimeter defines a concavity, and the optical cable is at least partially disposed in the concavity.

10. The UVC lighting system of claim 9, wherein the front and rear faces are defined by at least one pane of plexiglass.

11. The UVC lighting system of claim 10, wherein the front and rear faces are defined by a single pane of plexiglass.

12. The UVC lighting system of claim 1, wherein the substantially planar surface is defined by the outer sheath.

13. The UVC lighting system of claim 1, wherein the core comprises a polymeric optical fiber.

14. The UVC lighting system of claim 13, wherein the core comprises a single optical fiber.

15. The UVC lighting system of claim 13, wherein the core comprises a plurality of braided fibers.

16. The UVC lighting system of claim 13, wherein the outer sheath is co-extruded with the core.

17. The UVC lighting system of claim 1, wherein the optical cable defines a proximal end that is coupled to the optical source, and a distal end opposite the proximal end, the system further including an opaque cap that is configured to be coupled to the distal end so as to prevent the UVC light from being emitted from the distal end in a direction substantially parallel to the central axis.

18. The UVC lighting system of claim 1, wherein the UVC light source emits UVC light at a wavelength of approximately 222 nm.

19. The UVC lighting system of claim 1, wherein a substantial entirety of the UVC light emitted by the UVC light source is directed into the optical cable.

20. The UVC lighting system of claim 1, wherein the optical cable emits the UVC light in a direction substantially perpendicular to the central axis.
